(19) United States
(12) Patent Application Publication
(10) Pub. No.: US 2019/0047227 A1
(43) Pub. Date: Feb. 14, 2019

(54) 3D printing system based on multi-shaft linkage control and machine visual measurement

(71) Applicant: QUANZHOU INSTITUTE OF EQUIPMENT MANUFACTURING, Quanzhou, Fujian (CN)

(72) Inventors: Jun LI, Fujian (CN); Yinhui XIE, Fujian (CN); Jinxin LIN, Fujian (CN); Fengyu LI, Fujian (CN)

(21) Appl. No.: 15/738,138

(22) PCT Filed: Dec. 1, 2016
(86) PCT No.: PCT/CN2016/108183

§ 371 Date: Nov. 2, 2018

(30) Foreign Application Priority Data
Oct. 19, 2016 (CN) 2016109085767

(51) Int. Cl. B29C 64/393; B29C 64/112; B29C 64/227; B29C 64/209; A61F 2/28

(52) U.S. Cl. CPC B29C 64/393; B29C 64/112; B33Y 10/00; B29C 64/209; A61F 2/28; B29C 64/227

(57) ABSTRACT

A 3D printing system based on multi-shaft linkage control and machine vision measurement having a frame, a work table, a printing apparatus, a material conveying mechanism, image capturing cameras, a driving mechanism and a control system. The work table is a six degree-of-freedom linkage platform which is connected with the frame; the driving mechanism is in form of a six-axis robot arm; the printing apparatus is connected to the six-axis robot arm. The present 3D printing system is capable of achieving precise control of the spatial position of the printing nozzles of the printing apparatus.

### BACKGROUND OF THE INVENTION

The present invention relates to a 3D printing system based on multi-shaft linkage control and machine visual measurement and belongs to 3D printing field.

Currently, 3D printing methods for fabricating artificial bones mainly include stereolithography process, laminated object manufacturing, fused deposition modeling, selective laser sintering and inkjet deposition material additive modeling. Stereolithography process utilizes photosensitive resins which has poor biocompatibility and degradability and even induce toxic reaction after in vivo implantation. When fabricating artificial bones by laminated object manufacturing, materials such as hydroxyapatite have to be formed into curvable sheets, and the sheets are then glued together by a heated roller; from the perspective of material science, the aforesaid is difficult to implement. The printing materials used by fused deposition modeling have to undergo filament forming process and thus require a certain level of strength, thus resulting in limitations on selection of materials and difficulties for processing complex shapes. Selective laser sintering uses a laser system which is very expensive to purchase and maintain, resulting in higher production costs, and unsintered hydroxyapatite powders are hard to remove. Inkjet deposition material additive modeling may directly bind biocompatible aqueous solution and hydroxyapatite powders, thereby avoiding the strict requirements on printing conditions and temperatures of the aforementioned methods.

Existing 3D printing apparatus has complex nozzle structure and nozzle driving structure, and thus it is difficult to control printing precision. For example, in Chinese patent number CN 103948456 B the end of each of the nozzles are positioned on the same plane and move together and thus the nozzles occupy a larger space during operation; it is therefore not suitable for 3D printing of inner surface of porous structure.

In view of the foregoing, the inventor conducted in-depth research in respect of the aforementioned problems and devised the present invention.

### BRIEF SUMMARY OF THE INVENTION

An object of the present invention is to provide a 3D printing system based on multi-shaft linkage control and machine vision measurement which is simple in structure and is capable of enhancing printing precision.

To attain this, the present invention adopts the following technical scheme:

A 3D printing system based on multi-shaft linkage control and machine vision measurement which comprises a frame, a work table for receiving an artificial bone scaffold, a printing apparatus disposed on top of the work table, a material conveying mechanism for conveying printing materials, image capturing cameras, a driving mechanism for adjusting orientation of the printing apparatus and a control system; the printing apparatus, the material conveying mechanism, the image capturing cameras and the driving mechanism are all connected to the control system; the work table is a six degree-of-freedom linkage platform which is connected with the frame; the driving mechanism is in form of a six-axis robot arm; the printing apparatus is connected to the six-axis robot arm.

As a preferred embodiment of the present invention, the printing apparatus comprises a mounting frame, a motor, a pneumatic cylinder, a rotating flange and a plurality of nozzle assemblies; the mounting frame is connected to the six-axis robot arm; the motor is disposed on the mounting frame; the rotating flange is connected to an output shaft of the motor; the plurality of nozzle assemblies are evenly and circumferentially disposed on the rotating flange; the plurality of nozzle assemblies are connected to the mounting frame in a way which allows axial movement along the rotating flange; the pneumatic cylinder is disposed on the mounting frame; a driving end for driving movement of the nozzle assemblies is provided on a piston rod of the pneumatic cylinder; a reset mechanism for the nozzle assemblies to return to original position is provided between each of the nozzle assemblies and the rotating flange.

As a preferred embodiment of the present invention, the mounting frame comprises a protective cover, a first mounting board and a second mounting board; the first mounting board and the second mounting board are disposed at two axial ends of the protective cover respectively; the motor is fixedly disposed on the first mounting board; an operation opening is provided on the second mounting board for one of the nozzle assemblies to protrude outward; each of the nozzle assemblies has a top end which is pivotally connected to the rotating flange via a first connecting rod; the reset mechanism is in form of a first spring; the first spring has a first end which is connected to the rotating flange and a second end which is connected to the top end of the corresponding nozzle assembly; a mounting rod is further provided on the second mounting board; each of the nozzle assemblies further comprises a second connecting rod and a guiding cylinder; the second connecting rod has a first end which is pivotally connected to the mounting rod and a second end which is pivotally connected to the guiding cylinder; the nozzle assembly is slidingly movable inside the corresponding guiding cylinder.

As a preferred embodiment of the present invention, each of the nozzle assemblies comprises a cylindrical body and a movable piston disposed in an inner cavity of the cylindrical body; the movable piston divides the inner cavity of the cylindrical body into a first chamber and a second chamber; a printing nozzle is provided on the cylindrical body; the printing nozzle communicates with the second chamber; a gas inlet is provided on the cylindrical body; the gas inlet has a first end which communicates with the first chamber and a second end which communicates with a gas source; a material inlet is further provided; the material inlet has a first end which communicates with the second chamber and a second end which communicates with the material conveying mechanism.

As a preferred embodiment of the present invention, a piston stop is disposed inside the second chamber; the piston stop is disposed above the material inlet.

As a preferred embodiment of the present invention, there are 5 nozzle assemblies; the printing nozzles 4057 of the 5 nozzle assemblies are 120 um, 100 um, 80 um, 50 um and 30 um in diameter respectively.

As a preferred embodiment of the present invention, the material conveying mechanism comprises an air compressor and a material storing tank; an air outlet of the air compressor communicates with an air inlet of the material storing tank; a material outlet end of the material storing tank is connected to the material inlet of each of the nozzle assemblies via a material conveying tube; an electric valve is disposed at the material conveying tube.

As a preferred embodiment of the present invention, the frame comprises a base frame, a top frame, and a first side frame and a second side frame disposed between the base frame and the top frame; the six-axis robot arm is disposed on the top frame; the six degree-of-freedom linkage platform is disposed on the base frame; the six-axis robot arm and the six degree-of-freedom linkage platform are both connected to the control system.

As a preferred embodiment of the present invention, the image capturing cameras are disposed on the first side frame and/or the second side frame.

When the present invention is in use, the artificial bone scaffold is positioned on the six degree-of-freedom linkage platform. The position of the printing apparatus is controlled by the six-axis robot arm. With the coordination of the six degree-of-freedom linkage platform and the six-axis robot arm, precise control of the spatial position of the printing nozzles of the printing apparatus could be achieved, and 3D pattern printing of complex and fine artificial bone surface and inner surface of porous structure could be performed. The present invention has the advantages of simple in structure and high in printing precision. The present invention utilizes 3D printing technology based on inkjet deposition material additive modeling with nano-hydroxyapatite solution as printing material; motion redundancy of the robot arm is exploited to achieve 3D multi-angle printing and high-precision 3D pattern printing of complex and fine artificial bone surface and inner surface of porous structure. By means of precise mechanical motion control and control of droplet formation and dispersion via the six-axis robot arm and the six degree-of-freedom linkage platform, the printing precision of the system could reach ≤200 μm in printing surface resolution and ≤2 μm in layer resolution.

### BRIEF DESCRIPTION OF THE DRAWINGS

FIG. 1 is a schematic view showing the structure of the present invention.

FIG. 2 is a block diagram showing the control mechanism of the present invention.

FIG. 3 is a schematic view showing the structure of the printing apparatus of the present invention.

FIG. 4 is a schematic view showing the structure of the printing apparatus of the present invention, wherein the first mounting board and the protective cover are not shown.

FIG. 5 is an enlarged view of the portion “A” in FIG. 4.

FIG. 6 is a schematic view showing the structure of the printing nozzle of the present invention.

In the figures:

10 denotes the frame; 11 denotes the top frame; 12 denotes the base frame; 13 denotes the first side frame; 14 denotes the second side frame; 20 denotes the six-axis robot arm; 30 denotes the six degree-of-freedom linkage platform; 40 denotes the printing apparatus; 401 denotes the protective cover; 402 denotes the second mounting frame; 403 denotes the motor, 404 denotes the pneumatic cylinder; 405 denotes the nozzle assembly; 406 denotes the first connecting rod; 407 denotes the first spring; 408 denotes the mounting rod; 409 denotes the second connecting rod; 410 denotes the guiding cylinder; 411 denotes the rotating flange; 412 denotes the groove; 413 denotes the holder; 414 denotes the operation opening; 415 denote the first mounting board; 4050 denotes the cylindrical body; 4051 denotes the first chamber; 4052 denotes the second chamber; 4053 denotes the movable piston; 4054 denotes the sealing ring; 4055 denotes the conic guiding portion 4055; 4056 denotes the piston stop; 4057 denotes the printing nozzle; 4058 denotes the gas inlet; 4059 denotes the material inlet; 4060 denotes the electric valve; 50 denotes the image capturing cameras; 60 denotes the air compressor; 61 denotes the material storing tank; 62 denotes the air conveying tube; 63 denotes the material conveying tube; 70 denotes the control system.

### DETAILED DESCRIPTION OF THE INVENTION

The present invention is explained in further detail herein with the accompanying figures.

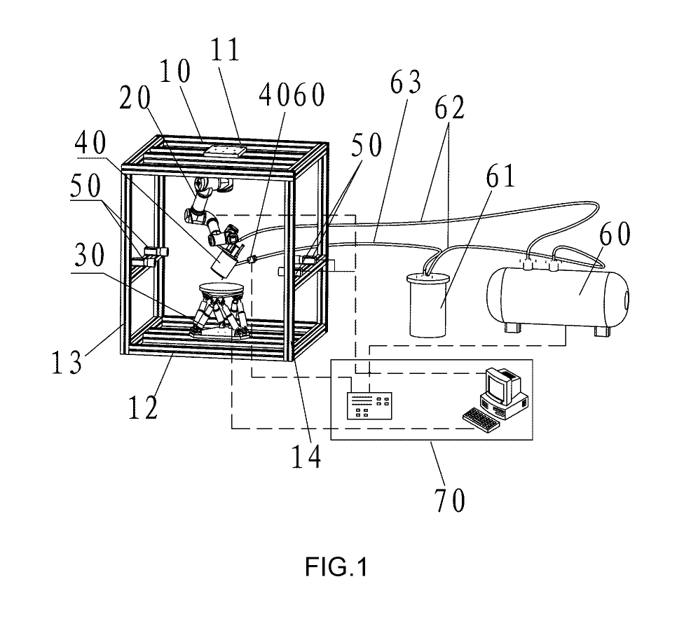

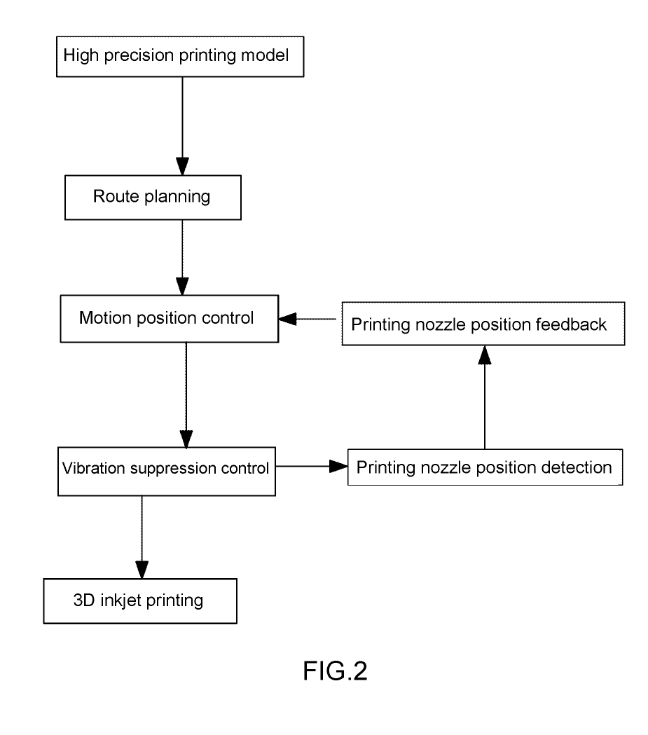

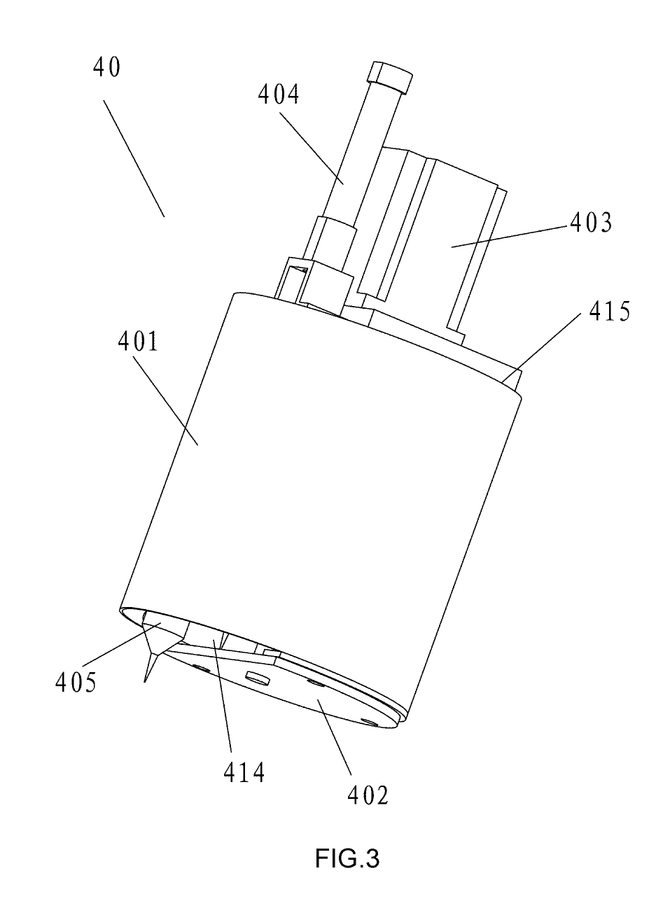

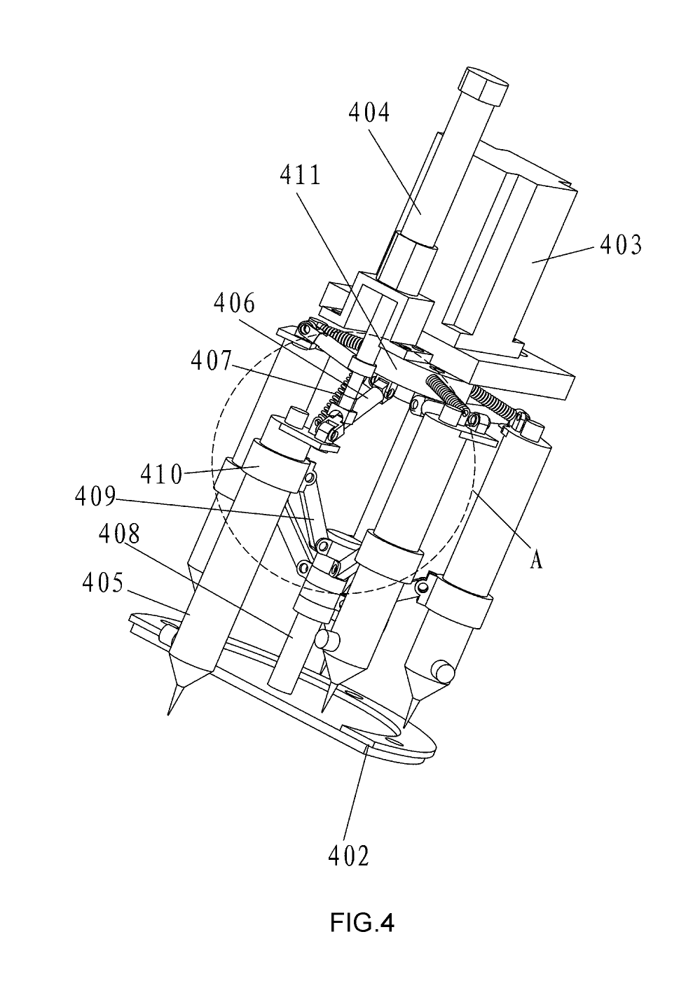

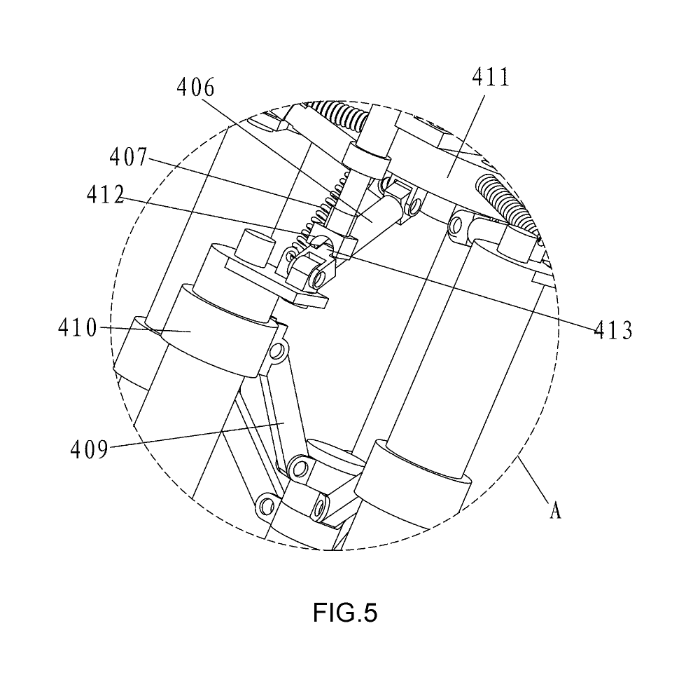

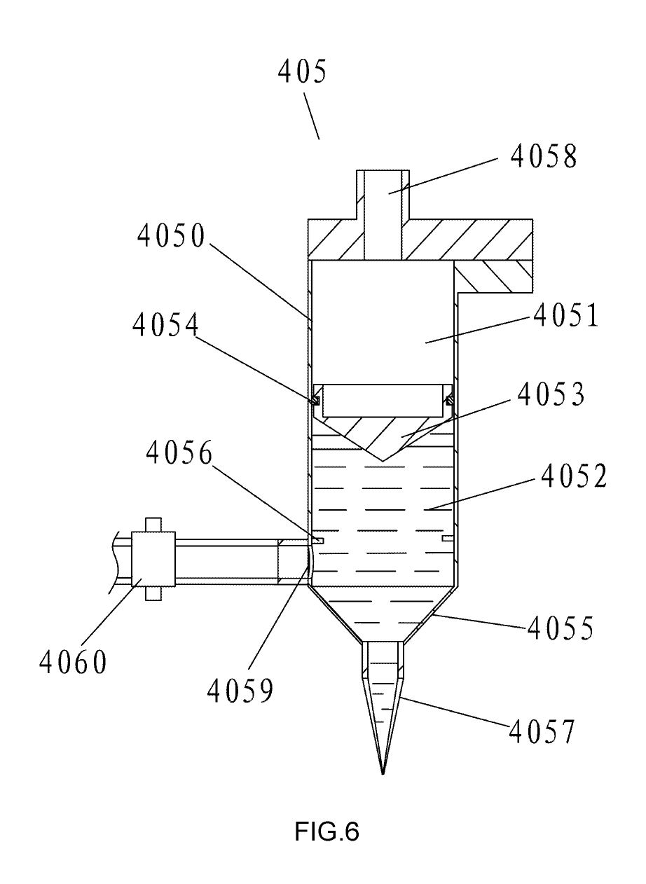

As illustrated in FIGS. 1-6, the 3D printing system based on multi-shaft linkage control and machine visual measurement comprises a frame 10, a work table for receiving an artificial bone scaffold, a printing apparatus 40 disposed on top of the work table, a material conveying mechanism for conveying printing materials, image capturing cameras 50, a driving mechanism for adjusting orientation of the printing apparatus 40 and a control system 70. The printing apparatus 40, the material conveying mechanism, the image capturing cameras 50 and the driving mechanism are all connected to the control system 70 which controls and coordinates operation of the aforesaid. The work table is a six degree-of-freedom linkage platform which is connected with the frame 10. The driving mechanism is in form of a six-axis robot arm 20. The printing apparatus 40 is connected to the six-axis robot arm 20. The control system 70 may be in form of the control and data processing system disclosed in the patent document titled “Automatic control turntable pneumatic multi-nozzle biological 3D printing forming system and method” with a patent number of CN 103948456 B. The control system 70 comprises a computer and a controller. The six-axis robot arm 20 is a six-axis robot arm which could achieve any conveying angle, and could be purchased directly from the marketplace. The six degree-of-freedom linkage platform 30 may adjust the space, position and orientation in six degree-of-freedom. It may be in form of the structure as disclosed in the patent document titled “Six degree-of-freedom linkage micro platform” with a patent number of CN 104002299 B, the details of which are not listed herein.

The 3D printing apparatus 40 comprises a mounting frame, a motor 403, a pneumatic cylinder 404, a rotating flange 411 and a plurality of nozzle assemblies 405. The mounting frame is connected to the six-axis robot arm 20. The motor 403 is disposed on the mounting frame. The rotating flange 411 is connected to an output shaft of the motor 403. The plurality of nozzle assemblies 405 are evenly and circumferentially disposed on the rotating flange 411. The plurality of nozzle assemblies 405 are connected to the mounting frame in a way which allows axial movement along the rotating flange 411. The pneumatic cylinder 404 is disposed on the mounting frame. A driving end for driving movement of the nozzle assemblies 405 is provided on a piston rod of the pneumatic cylinder 404. A reset mechanism for the nozzle assemblies 405 to return to original position is provided between each of the nozzle assemblies 405 and the rotating flange 411. In this embodiment, the output shaft of the motor 403, the pneumatic cylinder 404 and the nozzle assemblies 405 are arranged in parallel to each other. With the aforementioned structure, the motor 403 drives rotation of the rotating flange 411 and thereby rotate one of the nozzle assemblies 405 to a designated position; the pneumatic cylinder 404 then drives the nozzle assembly 405 to move axially so that it protrudes outward and perform 3D printing. After operation, that nozzle assembly 405 returns to its original position by means of the pneumatic cylinder 404 and the corresponding reset mechanism. The motor 403 then drives rotation of the rotating flange 411 to rotate another one of the nozzle assemblies 405 to a position corresponding to the pneumatic cylinder 404, and the pneumatic cylinder 404 then drives that nozzle assembly 405 to protrude outward. In this way, when the present invention performs 3D printing, the nozzle assembly 405 under operation does not lie in the same plane as other nozzle assemblies 405, thereby preventing other nozzle assemblies 405 from disturbing the artificial bone scaffold.

As a preferred embodiment of the present invention, the mounting frame comprises a protective cover 401, a first mounting board 415 and a second mounting board 402. The protective cover 401 is in cylindrical shape. The first mounting board 415 and the second mounting board 402 are fixedly disposed at two axial ends of the protective cover 401 respectively. The motor 403 is fixedly disposed on the first mounting board 415. The output shaft of the motor 403 passes through the first mounting board 415 and extends into the protective cover 401. An operation opening 414 is provided on the second mounting board 402 for one of the nozzle assemblies 405 to protrude outward. When one of the nozzle assemblies 405 is rotated to a position corresponding to the operation opening 414, the pneumatic cylinder 404 drives that nozzle assembly 405 to protrude outward from the operation opening 414 to perform printing. Each of the nozzle assemblies 405 has a top end which is pivotally connected to the rotating flange 411 via a first connecting rod 406. The reset mechanism is in form of a first spring 407. The first spring 407 has a first end which is connected to the rotating flange 411 and a second end which is connected to the top end of the corresponding nozzle assembly 405. A mounting rod 408 is further provided on the second mounting board 402. Each of the nozzle assemblies further comprises a second connecting rod 409 and a guiding cylinder 410. The second connecting rod 409 has a first end which is pivotally connected to the mounting rod 408 and a second end which is pivotally connected to the guiding cylinder 410. The nozzle assembly 405 is slidingly movable inside the corresponding guiding cylinder 410. Preferably, a groove 412 is provided on the piston rod of the pneumatic cylinder 404. A holder 413 which corresponds with the groove 412 is correspondingly disposed on the first connecting rod 406. When the piston rod of the pneumatic cylinder 404 protrudes to a certain position, the groove 412 rests on the holder 413 and drives the corresponding nozzle assembly 405 to move axially along the guiding cylinder 410. When the groove 412 leaves the holder 413, the nozzle assembly 405 moves along the guiding cylinder 410 under the action of the first spring 407, thereby achieving resetting of the nozzle assembly 405.

Each of the nozzle assemblies 405 of the 3D printing apparatus comprises a cylindrical body 4050 and a movable piston 4053 disposed in an inner cavity of the cylindrical body 4050. The movable piston 4053 divides the inner cavity of the cylindrical body 4050 into a first chamber 4051 and a second chamber 4052. A printing nozzle 4057 is provided on the cylindrical body 4050. The printing nozzle 4057 communicates with the second chamber 4052. A gas inlet 4058 is provided on the cylindrical body 4050. The gas inlet 4058 has a first end which communicates with the first chamber 4051 and a second end which communicates with a gas source. A material inlet 4059 is further provided. The material inlet 4059 has a first end which communicates with the second chamber 4052 and a second end which communicates with the material conveying mechanism. The present invention adopts inkjet deposition material additive modeling. The printing material is mainly nano-hydroxyapatite with the addition of collagen, chitin and other additives. The printing material is conveyed from the material inlet 4059 into the second chamber 4052. The movable piston 4053 is driven by the gas source to extrude the printing material from the printing nozzle 4057. The gas source is provided by an air compressor 60 as detailed below. Resetting of the movable piston 4053 is realized by feeding of material from the material inlet 4059.

As a preferred embodiment of the present invention, a piston stop 4053 is disposed inside the second chamber 4052. The piston stop 4053 is disposed above the material inlet 4059. Preferably, the cylindrical body 4050 forms a conic guiding portion 4055 at a lower end thereof. The printing nozzle 4057 is disposed on the conic guiding portion 4055. The material inlet 4059 is disposed between the piston stop 4053 and the printing nozzle 4057. Preferably, an annular groove is provided on a contact surface between the movable piston 4053 and the cylindrical body 4040. A sealing ring 4054 is provided in the annular groove.

As a preferred embodiment of the present invention, there are 5 nozzle assemblies. The printing nozzles 4057 of the 5 nozzle assemblies are 120 um, 100 um, 80 um, 50 um and 30 um in diameter respectively. The diameter sizes of the printing nozzles 4057 affect the printing precision and printing speed. Different diameter sizes of the printing nozzles 4057 may be selected according to printing needs. Switching of the printing nozzles 4057 is achieved by the motor 403, the rotating flange 411 and the pneumatic cylinder 404 in the present invention. It is easy to switch the printing nozzles 4057 and thus enhancing printing efficiency.

As a preferred embodiment of the present invention, the material conveying mechanism comprises the air compressor 60 and a material storing tank 61. An air outlet of the air compressor 60 communicates with an air inlet of the material storing tank 61 via an air conveying tube 62. A material outlet end of the material storing tank 61 is connected to the material inlets 4059 via a material conveying tube 63. An electric valve 4060 is disposed at the material conveying tube 63. With the aforementioned structure, printing material in the material storing tank 61 is conveyed to the nozzle assemblies 405 by air pressure.

As a preferred embodiment of the present invention, the frame 10 comprises a base frame 12, a top frame 11, and a first side frame 13 and a second side frame 14 disposed between the base frame 12 and the top frame 11. The six-axis robot arm 20 is disposed on the top frame 11. The six degree-of-freedom linkage platform 30 is disposed on the base frame 12. The six-axis robot arm 20 and the six degree-of-freedom linkage platform 30 are both connected to the control system 70. In this embodiment, image capturing cameras 50 are disposed on both the first side frame 13 and the second side frame 14.

In the present invention, the air compressor 60 controls the extrusion of printing material from the printing nozzles 4057 to achieve 3D printing. During printing, the air compressor 60 produces high pressure which is conveyed via the air conveying tube 62 to the gas inlet 4058 of the nozzle assembly 405 which protrudes outward to perform 3D printing. The sealing ring 4054 prevents air from entering the printing material. At the same time, the electric valve 4060 which is connected to the material inlets 4059 is at a closed state, thereby preventing printing material from discharging from the material inlets 4059. The movable piston 4053 of the nozzle assembly 405 which protrudes outward to perform 3D printing is driven by the high pressure air to move downwards to extrude printing material from the printing nozzle 4057 in a continuous and stable manner, thereby achieving 3D printing. When the movable piston 4053 moves to reach the piston stop 4056, the movable piston 4053 stops its movement. At the same time, the control system 70 controls the air compressor 60 to stop supplying air to the nozzle assembly 405. The control system 70 controls the electric valve 4060 which is connected to the material inlets 4059 to open, and controls the air compressor 60 to supply air to the material storing tank 61, thereby driving the printing material to enter the nozzle assemblies 405 via the material conveying tube 63. The movable piston 4053 moves towards the gas inlet 4058. When the printing material fills up the nozzle assemblies 405, the electric valve 4060 is closed, and the nozzle assemblies 405 continue printing. During printing, the extrusion speed of the printing material is controlled by controlling the air pressure, thereby achieving control precision.

Before performing 3D printing, the computer output of the present invention requires high precision printing model. The model is in STL format. After performing printing route planning, the multi-shaft linkage control system 70 controls coordination of the six-axis robot arm 20 and the six degree-of-freedom linkage platform 30 to achieve 3D pattern printing of complex and fine artificial bone surface and inner surface of porous structure. Motion models of the six-axis robot arm 20 and the six degree-of-freedom linkage platform 30 are built by computers, and motion routes thereof are optimized; the multi-shaft linkage control system 70 coordinates the motions of the six-axis robot arm 20 and the six degree-of-freedom linkage platform 30, and based on the overall moving speed of the six-axis robot arm 20, the printing droplet formation and dispersion are controlled, and the coordination of the six degree-of-freedom linkage platform 30 are controlled to perform 3D printing for complex surface. Multi-shaft linkage control could achieve high precision printing of 3D patterns for artificial bone surface and inner surface of porous structure and enhances printing efficiency. Furthermore, as the vibration of the six-axis robot arm 20 and the six degree-of-freedom linkage platform 30 during printing produces deviations and affects printing precision, the present invention provides vibration suppression control mechanism during motion process by building motion models for the six-axis robot arm 20 and the six degree-of-freedom linkage platform 30 respectively. With optimized control method based on linear binary pattern, linear binary control models for the six-axis robot arm 20 and the six degree-of-freedom linkage platform 30 are designed to control vibrations produced during system operation, thereby reducing the vibrations produced by the six-axis robot arm 20 and the six degree-of-freedom linkage platform 30 during the motion process, thus reducing printing deviations due to vibrations, and ensuring printing stability and enhancing printing precision. Two high-precision image capturing cameras 50 are installed on the first side frame 13 and the second side frame 14. The real-time position of the end of the printing nozzle 4057 is measured by binocular stereo vision; the spatial position of the end of the printing nozzle 4057 is obtained. By comparing the theoretical position and the measured real-time position of the end of the printing nozzle 4057, real-time feedback control of the position of the end of the printing nozzle 4057 could be achieved. Two high-precision image capturing cameras 50 obtain two images of the position of the end of the printing nozzle 4057 from different positions; by computing the position deviation of the end points of the printing nozzle 4057 in the two images, the spatial position of the end of the printing nozzle 4057 is obtained. The precision of the spatial position of the end of the printing nozzle 4057 obtained by the high-precision image capturing cameras 50 reaches μm level. Real-time feedback control of the position of the end of the printing nozzle 4057 is done based on the measured real-time position of the end of the printing nozzle 4057, and movement of the six-axis robot arm 20 is adjusted by computers, thereby ensuring high-precision printing.

The present invention provides improvements on 3D printing system, 3D printing apparatus and the nozzle assemblies of 3D printing apparatus. The form of the present invention does not limit to those as shown in the accompanying figures and the above embodiments. Any suitable changes or modifications based on similar principles should fall within the scope of the present invention.

What is claimed is:

1. A 3D printing system based on multi-shaft linkage control and machine vision measurement comprising a frame, a work table for receiving an artificial bone scaffold, a printing apparatus disposed on top of the work table, a material conveying mechanism for conveying printing materials, image capturing cameras, a driving mechanism for adjusting orientation of the printing apparatus and a control system; the printing apparatus, the material conveying mechanism, the image capturing cameras and the driving mechanism are all connected to the control system; characterized in that: the work table is a six degree-of-freedom linkage platform which is connected with the frame; the driving mechanism is in form of a six-axis robot arm; the printing apparatus is connected to the six-axis robot arm.

2. The 3D printing system based on multi-shaft linkage control and machine vision measurement as in claim 1, characterized in that: the printing apparatus comprises a mounting frame, a motor, a pneumatic cylinder, a rotating flange and a plurality of nozzle assemblies; the mounting frame is connected to the six-axis robot arm; the motor is disposed on the mounting frame; the rotating flange is connected to an output shaft of the motor; the plurality of nozzle assemblies are evenly and circumferentially disposed on the rotating flange; the plurality of nozzle assemblies are connected to the mounting frame in a way which allows axial movement along the rotating flange; the pneumatic cylinder is disposed on the mounting frame; a driving end for driving movement of the nozzle assemblies is provided on a piston rod of the pneumatic cylinder; a reset mechanism for the nozzle assemblies to return to original position is provided between each of the nozzle assemblies and the rotating flange.

3. The 3D printing system based on multi-shaft linkage control and machine vision measurement as in claim 2, characterized in that: the mounting frame comprises a protective cover, a first mounting board and a second mounting board; the first mounting board and the second mounting board are disposed at two axial ends of the protective cover respectively; the motor is fixedly disposed on the first mounting board; an operation opening is provided on the second mounting board for one of the nozzle assemblies to protrude outward; each of the nozzle assemblies has a top end which is pivotally connected to the rotating flange via a first connecting rod; the reset mechanism is in form of a first spring; the first spring has a first end which is connected to the rotating flange and a second end which is connected to the top end of the corresponding nozzle assembly; a mounting rod is further provided on the second mounting board; each of the nozzle assemblies further comprises a second connecting rod and a guiding cylinder; the second connecting rod has a first end which is pivotally connected to the mounting rod and a second end which is pivotally connected to the guiding cylinder; the nozzle assembly is slidingly movable inside the corresponding guiding cylinder.

4. The 3D printing system based on multi-shaft linkage control and machine vision measurement as in claim 3, characterized in that: each of the nozzle assemblies comprises a cylindrical body and a movable piston disposed in an inner cavity of the cylindrical body; the movable piston divides the inner cavity of the cylindrical body into a first chamber and a second chamber; a printing nozzle is provided on the cylindrical body; the printing nozzle communicates with the second chamber a gas inlet is provided on the cylindrical body; the gas inlet has a first end which communicates with the first chamber and a second end which communicates with a gas source; a material inlet is further provided; the material inlet has a first end which communicates with the second chamber and a second end which communicates with the material conveying mechanism.

5. The 3D printing system based on multi-shaft linkage control and machine vision measurement as in claim 4, characterized in that: a piston stop is disposed inside the second chamber; the piston stop is disposed above the material inlet.

6. The 3D printing system based on multi-shaft linkage control and machine vision measurement as in claim 5, characterized in that: there are 5 nozzle assemblies; the printing nozzles of the 5 nozzle assemblies are 120 um, 100 um, 80 um, 50 um and 30 um in diameter respectively.

7. The 3D printing system based on multi-shaft linkage control and machine vision measurement as in claim 6, characterized in that: the material conveying mechanism comprises an air compressor and a material storing tank; an air outlet of the air compressor communicates with an air inlet of the material storing tank; a material outlet end of the material storing tank is connected to the material inlets via a material conveying tube; an electric valve is disposed at the material conveying tube.

8. The 3D printing system based on multi-shaft linkage control and machine vision measurement as in claim 1, characterized in that: the frame comprises a base frame, a top frame, and a first side frame and a second side frame disposed between the base frame and the top frame; the six-axis robot arm is disposed on the top frame; the six degree-of-freedom linkage platform is disposed on the base frame; the six-axis robot arm and the six degree-of-freedom linkage platform are both connected to the control system.

9. The 3D printing system based on multi-shaft linkage control and machine vision measurement as in claim 8, characterized in that: the image capturing cameras are disposed on the first side frame and/or the second side frame.
